(19) United States
(12) Patent Application Publication
(10) Pub. No.: US 2026/0152960 A1
(43) Pub. Date: Jun. 4, 2026

(54) WATER QUALITY DEVICE DISPOSED IN A SKIMMER, FLOATING FILTER, OR WEIR COVER OF POOLS AND SPAS

(71) Applicants: Derek Shields, Provo, UT (US); Bryce Jenkins, Orem, UT (US); Alex Thompson, American Fork, UT (US)

(72) Inventors: Derek Shields, Provo, UT (US); Bryce Jenkins, Orem, UT (US); Alex Thompson, American Fork, UT (US)

(21) Appl. No.: 19/292,750

(22) Filed: Aug. 6, 2025

Related U.S. Application Data
(60) Provisional application No. 63/680,038, filed on Aug. 6, 2024.

(51) Int. Cl. E04H 4/12; C02F 1/00; C02F 1/68; C02F 103/42; G01N 33/18

(52) U.S. Cl. CPC E04H 4/1281; C02F 1/001; C02F 1/008; C02F 1/685; E04H 4/1263; G01N 33/1886; C02F 2103/42; C02F 2201/005; C02F 2201/006; C02F 2209/008; C02F 2209/02; C02F 2209/04; C02F 2209/06; C02F 2209/29; C02F 2209/40

(57) ABSTRACT

The present invention is a system and method for disposing a water sensing device in a pool or spa wherein the device may be disposed within a filter or skimming compartment in a sidewall of the pool or spa, or it may be disposed within a floating water skimming filter.

### FIELD OF THE INVENTION

This invention relates generally to water quality testing devices. More specifically, the invention pertains to advantageous positioning of a water quality testing sensor that can be conveniently disposed in a filtering device whether it is a mobile skimmer or a stationary location in the walls or structure of the pool or spa.

### DESCRIPTION OF RELATED ART

The prior art contains various water quality testing sensors that are capable of being used in pools and spas. However, these systems may be highly visible and ruin the aesthetics of the pool or spa. Other systems fail to adequately connect to the water system of the pool or spa.

Accordingly, it would be an advantage over the prior art to provide a water quality sensing system that can fit inside an existing enclosure of the pool or spa. It would be another advantage to provide a water quality sensing system that is adaptable to fit within an existing water skimmer or movable filtering system.

### BRIEF SUMMARY

The present invention is a system and method for disposing a water sensing device in a pool or spa wherein the device may be disposed within a filter or skimming compartment in a sidewall of the pool or spa, or it may be disposed within a floating water skimming filter.

In a first aspect of the invention, the water quality testing device is mounted inside the filter compartment so that it does not move and is free to test the water that passes through the compartment.

In a second aspect of the invention, the water quality testing device may also include a chemical dispensing system for dispensing chemicals into the water after testing.

In a third aspect of the invention, the water quality testing device may be disposed within a floating skimmer and perform water quality testing.

In a fourth aspect of the invention, the water quality testing device may be disposed within a floating skimmer and perform chemical dispensing.

These and other embodiments of the present invention will become apparent to those skilled in the art from a consideration of the following detailed description taken in combination with the accompanying drawings.

### BRIEF DESCRIPTION OF THE SEVERAL VIEWS OF THE DRAWINGS

FIG. 1 is a diagram of the first embodiment of the present invention disposed in a skimmer or filtering compartment.

FIG. 2 is a perspective view of a spa and showing the location of a filter housing built-in to the wall of the spa.

FIG. 3 is a perspective view of a different spa and shows the location of a filter housing built-in to the wall of the spa.

FIG. 4 is a perspective view of a different spa and shows the location of a filter housing built-in to the wall of the spa.

FIG. 5 is a perspective view of a different spa and shows the location of a filter housing built-in to the wall of the spa.

FIG. 6 is a perspective view of a different spa and showing the location of a filter housing built-in to the wall of the spa.

FIG. 7 is a perspective view of a different spa and shows the location of a filter housing built-in to the wall of the spa.

FIG. 8 is a perspective view of a different spa and shows the location of a filter housing built-in to the wall of the spa.

FIG. 9 is a perspective view of a different spa and shows the location of a filter housing built-in to the wall of the spa.

FIG. 10 is a perspective view of a different spa and showing the location of a filter housing built-in to the wall of the spa.

FIG. 11 is a perspective view of a second embodiment of the invention and shows in a cut-away view the components of the floating water sensing device.

FIG. 12 is a top view of a different spa and shows the location of a filter housing built-in to filters in the wall.

FIG. 13 is a close-up perspective view of the installed second embodiment.

### DETAILED DESCRIPTION

Reference will now be made to the drawings in which the various embodiments of the present invention will be discussed so as to enable one skilled in the art to make and use the invention. It is to be understood that the following description illustrates embodiments of the present invention and should not be viewed as narrowing the claims which follow.

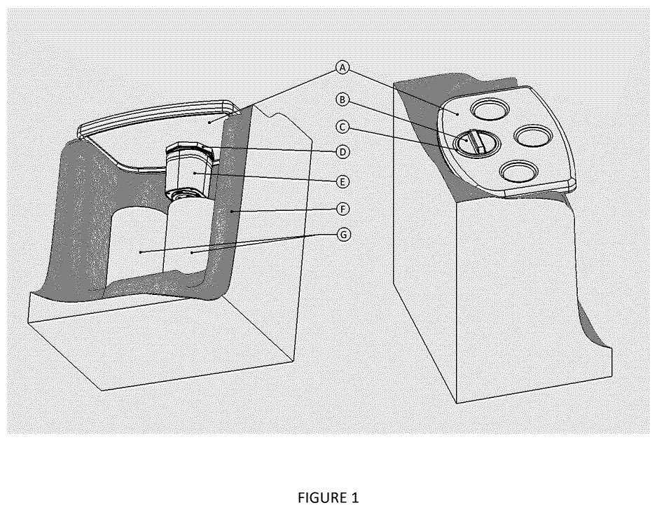

FIG. 1 is a diagram of the present invention disposed in a skimmer or filtering compartment. The Amano sensing device can test water from any location in a system where water is free of non-soluble contaminants. One such location is the skimmer or weir cover of pools and spas.

All pools and spas have a skimmer compartment of some sort which has a volume of water inside them. This spot is useful for placing the water sensing device as it allows the device to be in the water, but also have a place to mount the device without modifying the structure of the spa or pool itself.

FIG. 1 shows A) The lid or cover over the weir/skimmer compartment, B) A lid to cover the top of the device, C) A retaining ring which can be fitted into the lid of the skimmer compartment, a) This ring would be proprietary and in some cases different for different pools and spas, b) This ring locks into the top of the device as well as to the device lid, D) A nut which secures the retaining ring to the lid of the skimmer compartment, E) The Amono device, a) This would be a device identical to every other device, not a new SKU, F) The shell or wall of the pool or spa, G) The filters for the spa, a) In a pool this would be located in another location, like inside the pool house.

One of the main advantages of this placement of the water sensing device is that is in a physically stable location and hidden from view, and always contains water with no in-soluble contaminants.

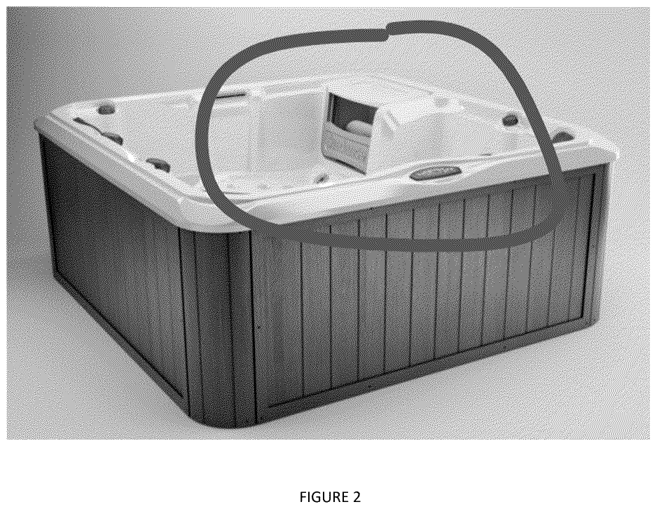

FIG. 2 is a perspective view of a spa and showing the location of a filter housing built-in to the wall of the spa. The first embodiment may be disposed within the filter housing.

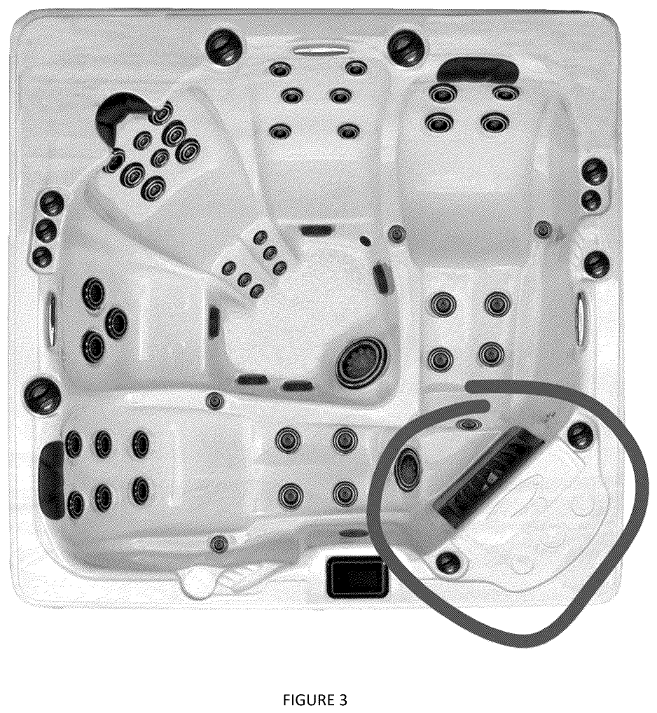

FIG. 3 is a perspective view of a different spa and shows the location of a filter housing built-in to the wall of the spa. The first embodiment may be disposed within the filter housing.

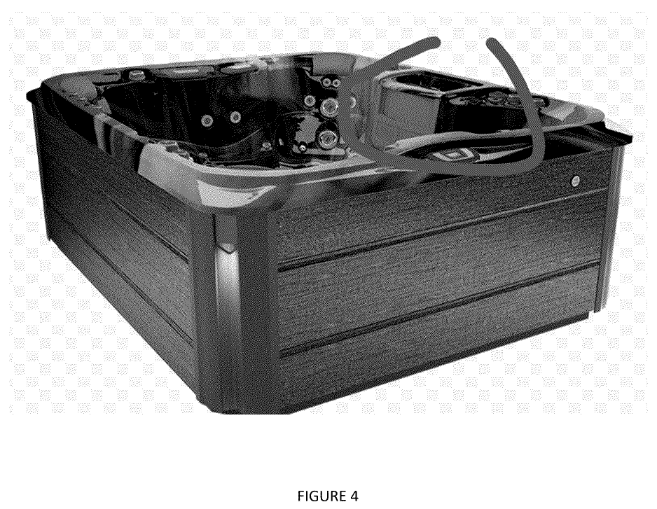

FIG. 4 is a perspective view of a different spa and shows the location of a filter housing built-in to the wall of the spa. The first embodiment may be disposed within the filter housing.

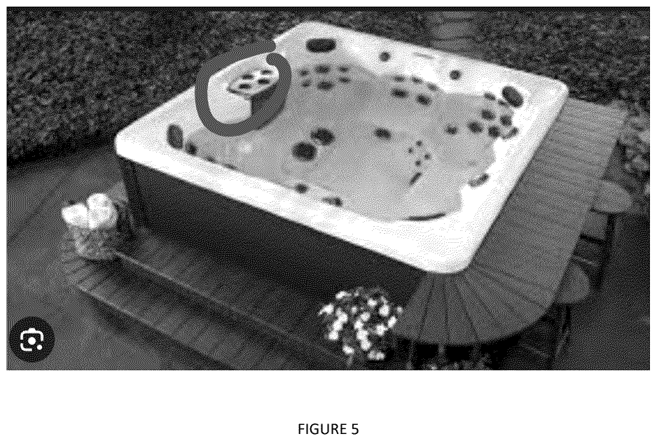

FIG. 5 is a perspective view of a different spa and shows the location of a filter housing built-in to the wall of the spa. The first embodiment may be disposed within the filter housing.

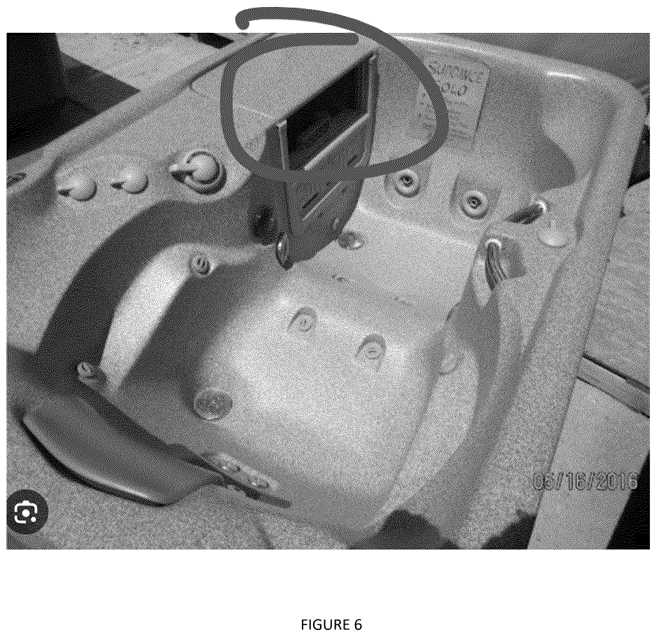

FIG. 6 is a perspective view of a different spa and showing the location of a filter housing built-in to the wall of the spa. The first embodiment may be disposed within the filter housing.

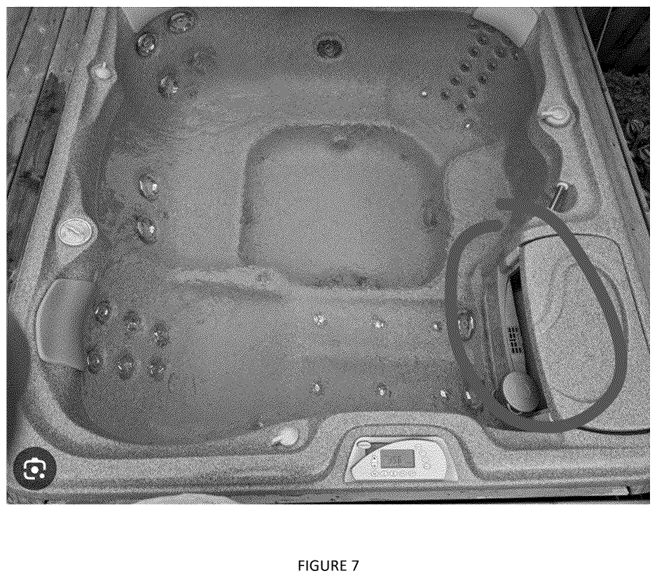

FIG. 7 is a perspective view of a different spa and shows the location of a filter housing built-in to the wall of the spa. The first embodiment may be disposed within the filter housing.

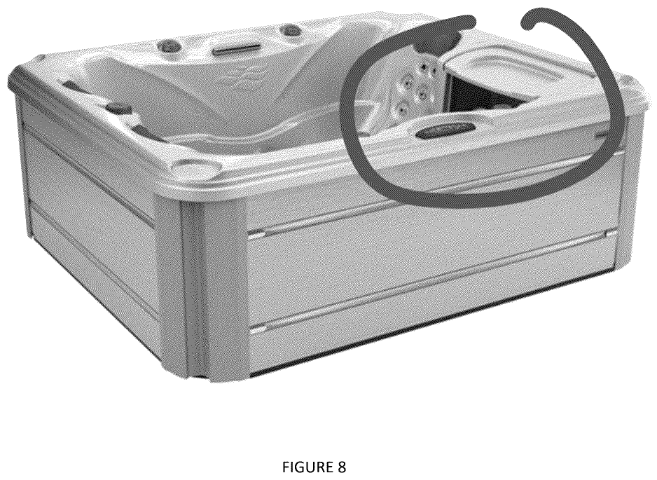

FIG. 8 is a perspective view of a different spa and shows the location of a filter housing built-in to the wall of the spa. The first embodiment may be disposed within the filter housing.

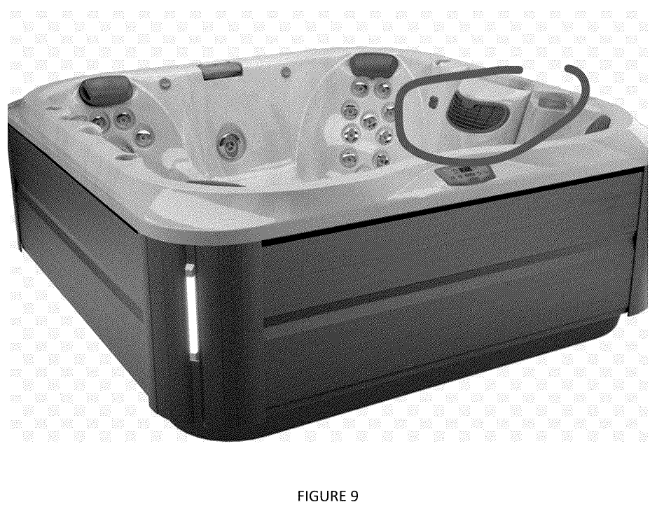

FIG. 9 is a perspective view of a different spa and shows the location of a filter housing built-in to the wall of the spa. The first embodiment may be disposed within the filter housing.

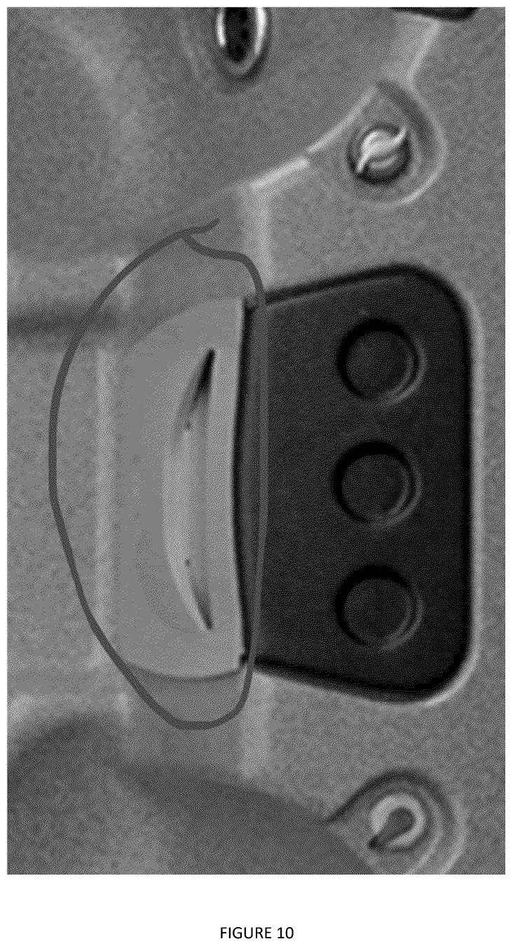

FIG. 10 is a perspective view of a different spa and showing the location of a filter housing built-in to the wall of the spa. The first embodiment may be disposed within the filter housing.

In a second embodiment of the invention, the water sensing device may be disposed inside a floating skimmer. Due to the size and floating nature of the device it is a good fit for a specific type of bobbing skimmer filters used in a lot of swim spas and pools. The original snap-on lid and bobber can be replaced with the Amano device and a custom bobber unit.

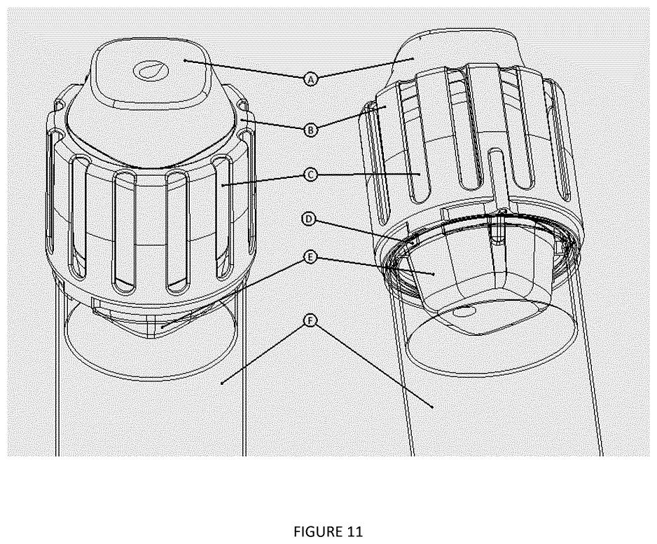

FIG. 11 shows in a cut-away perspective view the components of the floating water sensing device.

The water sensor includes A) Amano device lid, a) This is the current lid, no changes, B) Skimmer housing, a) This piece is proprietary and may change if different spas have different connection points, however it is likely most spas will have the same connection points, C) Amano Device, D) Retaining ring to secure the device encased in skimmer housing, E) Float assist for adding buoyancy to the device, F) Filter sleeve, and a) Supports the skimmer and device and adjusts with the level of the water in the spa to enable good flow through the spa's water circulation systems.

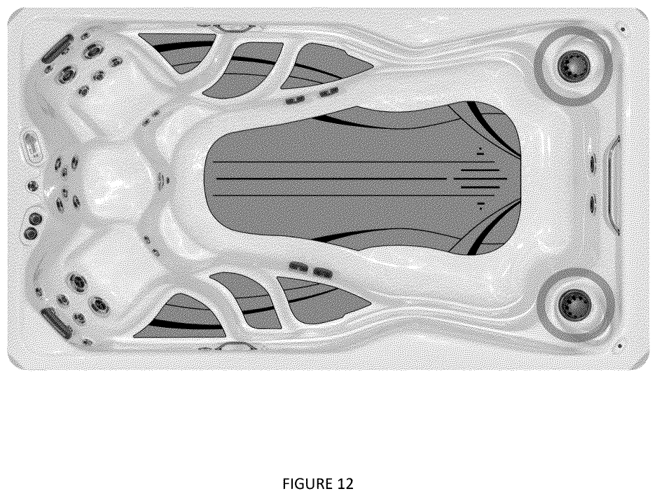

FIG. 12 is a top view of a different spa and shows the location of a filter housing built-in to filters in the wall in a second embodiment.

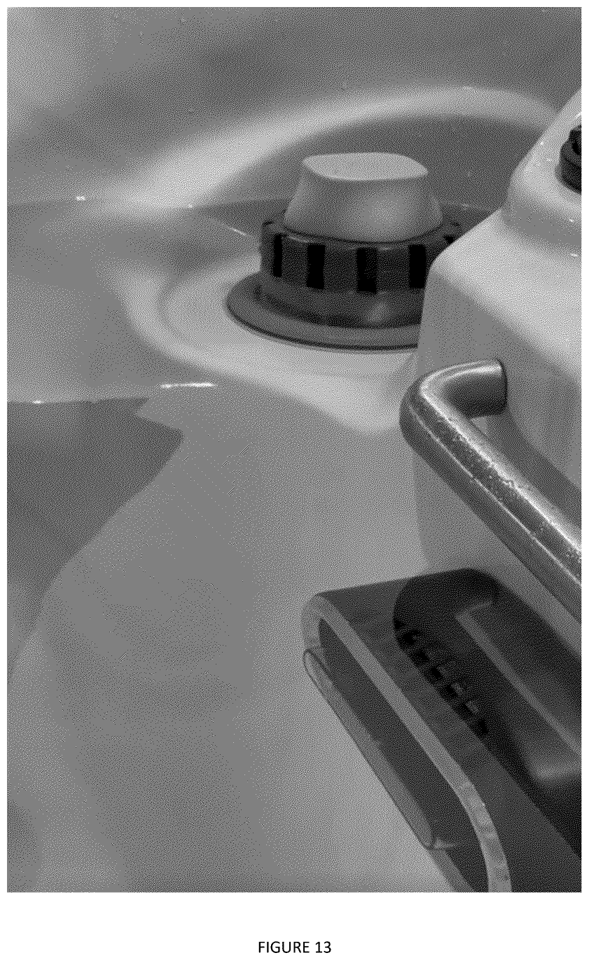

FIG. 13 is a close-up perspective view of the installed second embodiment.

In summary, the apparatus of a first embodiment may be described as a water-quality monitoring apparatus for a pool or spa, comprising: a sensor housing dimensioned to fit within an existing skimmer or filter compartment; a water-quality sensor disposed in the housing; a retaining ring engageable with a lid or cover of the compartment to releasably secure the housing in place; and control electronics configured to receive signals from the sensor and wirelessly transmit water-quality data.

Similarly, a summary of a method of use may be described as a method of monitoring and treating pool or spa water, comprising: positioning a water-quality monitoring apparatus inside a skimmer or filter compartment; sensing at least one water-quality parameter as water flows through the compartment; processing the sensed parameter to determine a water-quality condition; and wirelessly transmitting data indicative of the water-quality condition to a remote receiver.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. A water-quality monitoring apparatus for a pool or spa, comprising:
a sensor housing dimensioned to fit within an existing skimmer or filter compartment;
a water-quality sensor disposed in the housing;
a retaining ring engageable with a lid or cover of the compartment to releasably secure the housing in place; and
control electronics configured to receive signals from the sensor and wirelessly transmit water-quality data.

2. The apparatus of claim 1, further comprising a chemical-dispensing module carried by the housing and operable to meter treatment chemicals into the water.

3. The apparatus of claim 1, wherein the housing includes buoyancy elements sized to maintain the apparatus in a floating skimmer.

4. The apparatus of claim 1, wherein the water-quality sensor comprises at least one of a pH probe, an oxidation-reduction-potential probe, a chlorine probe, or a temperature sensor.

5. The apparatus of claim 1, wherein the control electronics include a battery power supply and a wireless transceiver selected from Bluetooth ® or Wi-Fi ®.

6. The apparatus of claim 2, wherein the chemical-dispensing module comprises a replaceable cartridge coupled to the housing.

7. The apparatus of claim 1, wherein the retaining ring is tightened to the lid by a threaded nut to allow tool-less installation and removal.

8. The apparatus of claim 3, wherein the buoyancy elements comprise a float-assist collar that maintains a predetermined vertical orientation of the sensor.

9. The apparatus of claim 1, further comprising a filter sleeve surrounding the housing and configured to permit water flow while screening debris from the sensor.

10. The apparatus of claim 1, wherein the housing includes interchangeable proprietary retaining rings sized for different skimmer geometries.

11. A method of monitoring and treating pool or spa water, comprising:
positioning a water-quality monitoring apparatus inside a skimmer or filter compartment;
sensing at least one water-quality parameter as water flows through the compartment;
processing the sensed parameter to determine a water-quality condition; and
wirelessly transmitting data indicative of the water-quality condition to a remote receiver.

12. The method of claim 11, further comprising dispensing a treatment chemical from the apparatus when the water-quality condition deviates from a predetermined range.

13. The method of claim 11, wherein positioning the apparatus includes locking a retaining ring of the apparatus to a lid of the compartment.

14. The method of claim 11, wherein positioning includes floating the apparatus within a floating skimmer while maintaining communication with the pool's circulation system.

15. The method of claim 11, further comprising powering the apparatus with an onboard battery and periodically waking the sensor to conserve power.

16. The method of claim 12, wherein dispensing the treatment chemical includes actuating a pump or valve to release a metered dose from a replaceable cartridge.

17. The method of claim 11, further comprising calibrating the sensor in situ by introducing a calibration solution through the compartment.

18. The method of claim 11, further comprising transmitting the data to a cloud service and generating a maintenance recommendation on a mobile device.

19. The method of claim 11, further comprising detecting a reduction in water flow through the compartment and sending an alert indicative of a potential filtration obstruction.
